(19) United States
(12) Patent Application Publication
(10) Pub. No.: US 2025/0232601 A1
(43) Pub. Date: Jul. 17, 2025

(54) SYSTEMS AND METHODS FOR AUTOMATED SAMPLE ANALYSIS

(71) Applicant: Testasy Inc., Allston, MA (US)

(72) Inventors: Michel Bielecki, Allston, MA (US); Loup Cordey, Allston, MA (US); Anna Kuzmina, Allston, MA (US); Javier Barranco Garcisa, Allston, MA (US); Jeyla Sadikova, Allston, MA (US)

(73) Assignee: Testasy Inc., Allston, MA (US)

(21) Appl. No.: 19/025,270

(22) Filed: Jan. 16, 2025

Related U.S. Application Data
(60) Provisional application No. 63/715,302, filed on Nov. 1, 2024.
(60) Provisional application No. 63/622,048, filed on Jan. 17, 2024.

(51) Int. Cl. G06V 20/69; G06T 7/00; G06V 10/141; G06V 10/82

(52) U.S. Cl. CPC G06V 20/698; G06T 7/0012; G06V 10/141; G06V 10/82; G06V 20/693; G06T 2207/30004

(57) ABSTRACT

Systems and techniques for automated sample analysis are described herein. In some embodiments, the system for automated sample analysis includes an imaging device for detecting optical signals encoded with information associated with a sample, a processor for generating a plurality of images of the sample based on the detected optical signals, and a machine learning model for determining an output indicative of one or more attributes of the sample based at least in part on the plurality of images of the sample. Data indicative of the one or more attributes of the sample may include: a presence of an inclusion in the sample, a size of an inclusion in the sample, a shape of an inclusion in the sample, a movement of an inclusion in the sample, and a number of an inclusion in the sample.

### CROSS-REFERENCE TO RELATED APPLICATIONS

This application claims priority to and the benefit under 35 U.S.C. § 119(e) of U.S. provisional patent application No. 63/622,048, filed on Jan. 17, 2024 under Attorney Docket No. T0930.70000US00, and titled “SYSTEMS AND METHODS FOR AUTOMATED SPERM ANALYSIS” and provisional patent application No. 63/715,302, filed on Nov. 1, 2024 under Attorney Docket No. T0930.70000US01, and titled “SYSTEMS AND METHODS FOR AUTOMATED SAMPLE ANALYSIS,” both for which the entire contents are hereby incorporated by reference herein.

### SUMMARY OF THE DISCLOSURE

In some aspects, it is appreciated that improved methods for performing health assessments and determining fertility issues in men are needed. In particular, current conventional methods and technology consist of complicated, labor intensive, high cost, and laboratory-centric instruments and methodologies. In some aspects, various systems and methods are provided herein that provide low-cost, streamlined, and technological solutions for performing automated sperm analysis. In some implementations, such solutions can be implemented without the need for specialized laboratory equipment, highly trained specialists, or outsourcing the testing to an expensive and specialized laboratory. Further, while the systems and techniques are described herein primarily with respect to sperm analysis, it can be appreciated that the systems and techniques can be used for other biological analysis and non-biological analysis applications such as, for example, urinalysis, pleural fluid microscopic analysis, joint fluid microscopic analysis, ascites fluid microscopic analysis, water quality analysis, industrial particle size distribution analysis, or any other suitable analysis.

According to some aspects of the technology described herein, an imaging system for automated sperm analysis is provided. The imaging system comprises: an imaging device configured to detect optical signals encoded with information associated with a sperm sample; a processor operatively coupled with the imaging device and configured to receive the detected optical signals and generate a plurality of images of the sperm sample based at least in part on the detected optical signals; and a machine learning model configured to receive the plurality of images of the sperm sample as input from the processor and determine an output indicative of a health of the sperm sample based at least in part on the received plurality of images of the sperm sample.

In some embodiments, the machine learning model is trained at least in part using training images having a higher resolution than a resolution of the received plurality of images. In some embodiments, the data indicative of the health of the sperm sample comprises an indication of whether sperm in the sperm sample is a normal shape.

In some embodiments, the machine learning model is an adversarial neural network comprising: a first portion, trained using the training images having the higher resolution, configured to generate data indicative of the health of the sperm sample based on the received plurality of images of the sperm sample; and a second portion configured to generate new data indicative of the health of the sperm sample based at least in part on the data generated by the first portion; and the output is determined based at least on the data generated by the first portion and the new data generated by the second portion.

In some embodiments, the machine learning model is an adversarial neural network comprising: a first portion, trained using the training images having the higher resolution, configured to extract images' features based on the received plurality of images of the sperm sample; a second portion that generates data indicative of the health of the sperm sample based on the features received from the first portion; and a third portion configured to generate new data indicative of the health of the sperm based at least in part on the data generated by the first portion; and the output is determined based at least on the data generated by the second portion and the new data generated by the third portion.

In some embodiments, the machine learning model is a deep learning model configured to determine data indicative of the health of the sperm sample. In some embodiments, the data indicative of the health of the sperm sample includes at least one of: concentration of the sperm sample and motility of the sperm sample. In some embodiments, motility of the sperm sample comprises data indicative of the motion of at least one sperm in the sperm sample.

In some embodiments, the machine learning model is a first machine learning model configured to determine at least one of a concentration of the sperm sample and a motility of the sperm sample; the system further comprises a second machine learning model configured to determine whether sperm in the sperm sample is a normal shape; and the output indicative of a health of the sperm sample includes a first determination from the first machine learning model and a second determination from the second machine learning model.

In some embodiments, the imaging device comprises: an emitter comprising a light source configured to illuminate the sperm sample; a sample holder to hold the sperm sample; and a sensor configured to detect the optical signals encoded with information associated with the sperm sample.

In some embodiments, the light source is a first light source of a plurality of light sources arranged in an array and is configured to vary at least one illumination condition of the plurality of light sources. In some embodiments, The system of claim 10, wherein the emitter is configured to operate in a pulse mode and operation of the emitter is configured to be synchronized with operation of the sensor.

In some embodiments, at least two of the imaging device, processor, and machine learning model are integrated on the same device.

In some embodiments, the optical signals detected by the imaging device are a first set of optical signals associated with a first illumination condition of the imaging device; and the imaging device is further configured to detect a second set of optical signals associated with a second illumination condition of the imaging device. In some embodiments, generating the plurality of images comprises generating a plurality of high-resolution images based at least in part on the first and second sets of optical signals.

According to some aspects of the technology described herein, a method for automated sperm analysis is provided. The method comprises: detecting, with an imaging device, optical signals encoded with information associated with a sperm sample; generating a plurality of images of the sperm sample based at least in part on the optical signals; and determining, using a machine learning model, an output indicative of a health of the sperm sample based at least in part on the generated plurality of images of the sperm sample.

In some embodiments, detecting optical signals encoded with information associated with the sperm sample comprises: illuminating the sperm sample using an emitter of the imaging device, the emitter disposed on a first side of the sperm sample; and detecting the optical signals with an optical sensor the imaging device, the optical sensor disposed on a second side of the sperm sample opposite the first side.

In some embodiments, determining the output indicative of the health of the sperm sample using the machine learning model comprises: generating, using a first portion of the machine learning model, data indicative of the health of the sperm sample based on the plurality of images of the sperm sample; and generating, using a second portion of the machine learning model, new data indicative of the health of the sperm sample based at least in part on the data generated by the first portion.

In some embodiments, generating the plurality of images of the sperm sample comprises: determining first amplitude values and first phase values for the detected optical signals at a first plane of the sensor; determining second amplitude values and second phase values for the detected optical signals at a second plane of the sperm sample based on the first amplitude values and first phase values; and updating the first amplitude values and first phase values at the first plane based on the second amplitude values and second phase values.

In some embodiments, generating the plurality of images comprises: identifying, using a second machine learning model, one or more artifacts present in the plurality of images; and removing, using the second machine learning model, the identified one or more artifacts from the plurality of images.

According to some aspects of the technology described herein, an imaging system for automated sample analysis is provided. The imaging system comprises: an imaging device configured to detect optical signals encoded with information associated with a sample, the imaging device comprising: an emitter comprising a light source configured to illuminate the sample; a sensor configured to detect the optical signals encoded with information associated with the sample; and a sample holder disposed between the light source and the sensor, the sample holder configured to hold the sample and allow light to pass through from the light source to the sensor; a processor operatively coupled with the sensor of imaging device and configured to receive data indicative of the detected optical signals and generate a plurality of images of the sample based at least in part on the detected optical signals; and a machine learning model configured to receive the plurality of images of the sample as input from the processor and determine an output indicative of one or more attributes of the sample based at least in part on the received plurality of images of the sample.

In some embodiments, the machine learning model is trained at least in part using training images having a higher resolution than a resolution of the received plurality of images. In some embodiments, the one or more attributes comprise at least one of: a presence of an inclusion in the sample, a size of an inclusion in the sample, a shape of an inclusion in the sample, a movement of an inclusion in the sample, and a number of an inclusion in the sample.

In some embodiments, the machine learning model is an adversarial neural network comprising: a first portion, trained using the training images having the higher resolution, configured to generate data indicative of the one or more attributes of the sample based on the received plurality of images of the sample; and a second portion configured to generate new data indicative of the one or more attributes of the sample based at least in part on the data generated by the first portion; and the output is determined based at least on the data generated by the first portion and the new data generated by the second portion.

In some embodiments, the machine learning model is an adversarial neural network comprising: a first portion, trained using the training images having the higher resolution, configured to extract image features based on the received plurality of images of the sample; a second portion configured to generate data indicative of the one or more attributes of the sample based on the features received from the first portion; and a third portion configured to generate new data indicative of the one or more attributes of the sample based at least in part on the data generated by the first portion; and the output is determined based at least on the data generated by the second portion and the new data generated by the third portion.

In some embodiments, the machine learning model is a deep learning model configured to determine the one or more attributes of the sample. In some embodiments, the machine learning model is a first machine learning model configured to determine at least a first attribute of the one or more attributes of the sample; the system further comprises a second machine learning model configured to determine at least a second attribute of the one or more attributes of the sample; and the output includes a first determination from the first machine learning model and a second determination from the second machine learning model.

In some embodiments, at least two of the imaging device, processor, and machine learning model are integrated on the same device. In some embodiments, the light source is a first light source; the emitter comprises a plurality of light sources arranged in an array; and the emitter is configured to vary at least one illumination condition of the plurality of light sources.

In some embodiments, the optical signals detected by the imaging device are a first set of optical signals associated with a first illumination condition of the plurality of light sources; and the imaging device is further configured to detect a second set of optical signals associated with a second illumination condition of the plurality of light sources.

In some embodiments, generating the plurality of images comprises generating a plurality of high-resolution images based at least in part on the first and second sets of optical signals. In some embodiments, each of at least a subset of the plurality of images is associated with a respective depth of the sperm sample.

In some embodiments, the emitter is configured to operate in a pulse mode and operation of the emitter is configured to be synchronized with operation of the sensor. In some embodiments, the operation of the emitter is synchronized with the operation of the sensor by receiving, with the emitter, one or more signals from the sensor.

According to some aspects of the present technology, a method for automated sample analysis is provided. The method comprises: detecting, with an imaging device, optical signals encoded with information associated with a sample by: illuminating the sample using an emitter of the imaging device, the emitter disposed on a first side of the sample; and detecting the optical signals with a sensor of the imaging device, the sensor disposed on a second side of the sample opposite the first side; generating a plurality of images of the sample based at least in part on the optical signals; and determining, using a machine learning model, an output indicative of one or more attributes of the sample based at least in part on the generated plurality of images of the sample.

In some embodiments, generating the plurality of images of the sample comprises: determining first amplitude values and first phase values for the detected optical signals at a first plane of the sensor; determining second amplitude values and second phase values for the detected optical signals at a second plane of the sample based on the first amplitude values and first phase values; and updating the first amplitude values and first phase values at the first plane based on the second amplitude values and second phase values.

In some embodiments, generating the plurality of images comprises: identifying, using a second machine learning model, one or more artifacts present in the plurality of images; and removing, using the second machine learning model, the identified one or more artifacts from the plurality of images.

In some embodiments, determining the output indicative of one or more attributes of the sample using the machine learning model comprises: generating, using a first portion of the machine learning model, data indicative of the one or more attributes of the sample based on the plurality of images of the sample; and generating, using a second portion of the machine learning model, new data indicative of the one or more attributes of the sample based at least in part on the data generated by the first portion.

In some embodiments, detecting the optical signals comprises: illuminating the sample with two or more illumination conditions of the imaging device; and detecting optical signals associated with each illumination condition of the two or more illumination conditions.

In some embodiments, generating the plurality of images of the sample comprises: matching first features of the detected optical signals associated with a first illumination condition with second features of the detected optical signals associated with a second illumination condition; and generating the plurality of images of the sample based on the matched first features and second features.

### BRIEF DESCRIPTION OF THE DRAWINGS

Various aspects of at least one embodiment are discussed herein with reference to the accompanying figures, which are not intended to be drawn to scale. The figures are included to provide illustration and a further understanding of the various aspects and embodiments and are incorporated in and constitute a part of this specification but are not intended as a definition of the limits of the invention. Where technical features in the figures, detailed description or any claim are followed by reference signs, the reference signs have been included for the sole purpose of increasing the intelligibility of the figures, detailed description, and/or claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure.

FIG. 1 depicts an exemplary method for automated sperm analysis, according to some embodiments.

FIG. 2A depicts an exemplary embodiment of a diagnostic system for automated sperm analysis, according to some embodiments.

FIG. 2B depicts various exemplary configurations of an imaging device for a diagnostic system for automated sperm analysis, according to some embodiments.

FIG. 2C depicts a graphical representation of an electronic rolling shutter (ERS) image acquisition scheme, according to some embodiments.

FIG. 2D depicts a graphical representation of a global reset release (GRR) image acquisition scheme, according to some embodiments.

FIG. 3A depicts an exemplary computational flow for reconstructing an image of a sperm sample captured by an imaging device, according to some embodiments.

FIG. 3B depicts an exemplary computation flow for reconstructing a high-resolution image of a sperm sample based on various detected optical signals of the sperm sample, according to some embodiments.

FIG. 4 depicts an exemplary machine learning model for determining an indication of the health or quality of a sperm sample based at least in part on a received 2D or 3D image, according to some embodiments.

FIGS. 5A-5B depict exemplary outputs of the model including attributes indicating the health or quality of a sperm sample, according to some embodiments.

FIGS. 6A-6D depict exemplary outputs of a model including path tracking of a sperm sample, according to some embodiments.

FIG. 7 depicts an exemplary configuration of an imaging device for a diagnostic system for automated sample analysis, according to some embodiments.

FIG. 8 depicts an exemplary method for automated sample analysis, according to some embodiments.

FIG. 9 depicts images of one or more inclusions that may be evaluated and identified in a sample using the systems and techniques described herein, according to some embodiments.

FIG. 10 depicts a non-limiting group of crystalline precipitates that may be present in a urine sample.

### DETAILED DESCRIPTION

It is appreciated that current diagnostic approaches for reproductive health and fertility issues focus primarily on women, as do current advancements in the diagnostic technology and methods. This is contrary to the fact that men contribute approximately 50% to fertility issues seen among the population. Apart from the reproductive and fertility issues, sperm quality can be an important indicator in understanding male health and can be linked to hormone issues, vascular problems, heart disease, prostate cancer, longevity and other aspects of male health. As such, early and routine testing may be a key component of preventative healthcare for men.

The inventors have recognized and appreciated that conventional diagnostic methods and technologies focusing on male reproductive health and fertility issues, however, face a number of shortcomings that may make them difficult and inaccessible for both fertility and reproductive uses as well as preventative healthcare uses. Conventional methods and technology consist of complicated, labor intensive, high cost, and laboratory-centric instruments and methodologies. Conventional techniques typically require physicians with specialized training to conduct long and specialized analysis processes, many parts of which may be conducted manually by the specially-trained physician. This requires a medical facility to either have a physician who has gone through the specialized training on hand or send the analysis to a fertility testing laboratory that can routinely conduct this testing, increasing both time and cost of the testing. While some improvements have been made to male fertility testing through computer assisted analysis processes, these methods utilize conventional processing techniques that are less accurate and reliable than their human-conducted counterparts.

Accordingly, as described herein, the inventors have developed methods and systems for providing medical offices low-cost, streamlined, and technological solutions that can be placed in any medical office without the need for specialized laboratory equipment, trained specialists, or outsourcing the testing to an expensive and specialized laboratory. By leveraging artificial intelligence (AI) and machine learning capabilities with advancements in sample imaging technologies, the inventors have developed reliable and accurate technologies and methods for male fertility testing that can contribute to fertility and reproductive healthcare as well as general preventative healthcare for men.

Further, the inventors have recognized and appreciated that the methods and systems described herein can provide similar benefits for biological and non-biological analyses other than automated sperm analysis. For example, in some embodiments, the systems and techniques may be used to perform one or more aspects of a typical urinalysis, which can reduce the cost, complexity, and specialized training needed to perform these aspects of the analysis. In other embodiments, the systems and techniques may be used to perform a water quality assessment in determining the presence of bacteria, protozoa, or other water contaminants in a water sample.

The systems and techniques described herein may be used for any suitable analysis, including but not limited to: automated sperm analysis, urinalysis, hematology analysis, microbiological and cell biology analyses, biological fluid analyses (cerebrospinal, thoracentesis, pleural fluid, joint fluid, etc.), and biopsy analysis (mass, urological, prenatal, etc.), the details of which will be described further herein.

According to some aspects described herein, a method for automated sperm analysis may be provided. The method may start by detecting optical signals encoded with information associated with a sperm sample. For example, the detected optical signals may be encoded with information indicative of how the sperm sample interacts with incoming light from a light source. In some embodiments, this information may be determined from how light interacting with the sperm sample interacts with incoming light from a light source. In some embodiments, the optical signals detected may encode information about the sperm sample for a given depth of the sample. In some embodiments, the optical signals may encode information associated with the entire sperm sample, for example, at a plurality of depths throughout the sample. The number of layers of the sperm sample which the optical signals may be associated with may include any number of depths, for example 5 different depths, 10 different depths, or any suitable number of depths. In some embodiments, the number of depths may be determined by the parameters set by an algorithm configured to reconstruct the detected optical signals.

The detected optical signals may then be used to generate a plurality of images of the sperm sample. In some embodiments, a two-dimensional (2D) image may first be reconstructed using the optical signals encoded with information associated with the sperm sample at a specific depth. Repeating the reconstruction method using optical signals encoded with information associated with the sperm sample at various different depths may be used to generate a plurality of 2D images of the sperm sample each at a respective depth and may be used to generate a three-dimensional (3D) representation of the sperm sample. Details about reconstructing the 2D image will be described further herein.

After generating the plurality of images, the images may then be analyzed to determine an indication of the health and quality of the sperm sample. For example, the indication of the health and quality of the sperm sample may include one or more calculated metrics assessing the health and quality of the sperm sample, including but not limited to concentration of the sperm sample, motility of the sperm sample, morphology of at least one sperm in the sperm sample. In some examples, the indication of the health and quality of the sperm sample may include a measure of the motion of one or more sperm in the sperm sample along either or both of a 2D path or 3D path. In some examples, the analysis may be done by using the 2D or 3D image as input to a machine learning model configured to determine one or more attributes of a particular sample, for example, one or more of the various metrics discussed above to determine an indication of the health and quality of the sperm sample. The one or more attributes may additionally or alternatively include the presence of an inclusion in the sample, the size, shape, or morphology of an inclusion in the sample, the number of a particular inclusion in the sample (e.g., concentration, number per imaged sample area, number per high or low powered field), or any other suitable attribute.

With respect to sperm analysis, in some embodiments, the machine learning model may be a first machine learning model configured to determine indications of the health and quality of the sperm including but not limited to attributes such as concentration, motility, and motion tracking. In some embodiments, a second machine learning model may be configured to determine indications of the health and quality of the sperm including but not limited to morphology of the sperm sample.

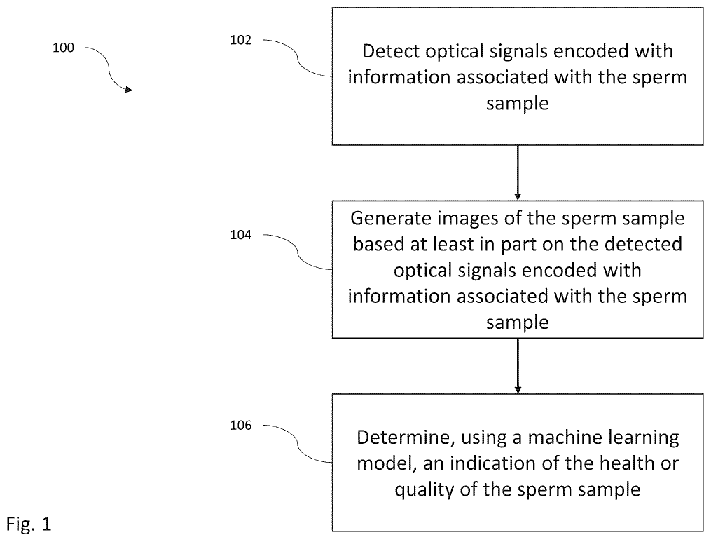

FIG. 1 depicts an exemplary method 100 for automated sperm analysis, according to some embodiments. The method for automated sperm analysis may start, at step 102, by detecting optical signals encoded with information associated with the sperm sample. For example, an imaging device (e.g., an inline holographic microscopy device) may be configured to illuminate the sample with a light source and detect the optical signals that pass through the sperm sample.

At step 104, the images of the sperm sample may be generated based at least in part on the detected optical signals encoded with information associated with the sperm sample. In some embodiments, each of the images may be associated with a different depth of the sperm sample which can be stacked to generate a 3D representation of the sperm sample. In some embodiments, a first set of images may be associated with the sperm sample at a first point in time and a second set of images may be associated with the sperm sample at a second point in time later than the first. Multiple sets of images associated with the sperm sample over a period of time may be used to generate a video of the sperm sample over the period of time rather than a static image.

At step 106, the generated images may then be analyzed to determine an indication of the health or quality of the sperm sample, for example, by determining one or more metrics of the sperm sample. In some embodiments, the one or more metrics may include concentration of the sperm sample, motility of the sperm sample, morphology of the sperm sample, or the path that the sperm in the sperm sample may move along. In some embodiments, the analysis and determination may be done by a machine learning model as described further herein.

According to some aspects described herein, a diagnostic system for automated sperm analysis may be provided. The diagnostic system may implement the method described above or any other method described herein. In some examples, the diagnostic system may include an imaging device configured to detect optical signals encoded with information associated with the sperm sample. The optical signals may be encoded with information associated with the sperm sample at a given depth or may be associated with the sperm sample at a plurality of depths. The number of layers of the sperm sample which the optical signals may be associated with may include any number of depths, for example 5 different depths, 10 different depths, or any suitable number of depths. In some embodiments, the number of depths may be determined by the parameters set by an algorithm configured to reconstruct the detected optical signals. The imaging device may detect optical signals encoded with information about the sperm sample at a given time. In some embodiments, the imaging device may detect optical signals encoded with information about the sperm sample over a period of time to be able to reconstruct certain metrics regarding the motion of the sperm in the sperm sample.

The diagnostic system may further include a processor configured to receive the detected optical signals and generate a plurality of images of the sperm sample. As described with respect to the method above, a two-dimensional (2D) image may first be reconstructed using the optical signals encoded with information associated with the sperm sample at a specific depth. Repeating the reconstruction method using optical signals encoded with information associated with the sperm sample at various different depths may be used to generate a plurality of 2D images of the sperm sample each at a respective depth and may be used to generate a three-dimensional (3D) representation of the sperm sample. In some embodiments, the plurality of images may be a first plurality of images associated with the sperm sample at a given point in time. In some embodiments, the processor may generate a second plurality of images associated with the sperm sample at a second time or may generate various pluralities of images associated with the sperm sample over a period of time to create a video of the sperm sample. As discussed above, the processor may be implemented as a CPU, GPU, or any other suitable processing component. In some embodiments, the processing device may be disposed separately from but operatively coupled to (e.g., over a communication network) the diagnostic system. For example, the processing device may be part of a cloud computing system or a separate but operatively coupled device. Details on the method of reconstruction according to some embodiments is discussed further herein.

The diagnostic system may further include a machine learning model configured to receive the plurality of images from the processor and determine an indication of the health and quality of the sperm sample based at least in part on the plurality of images. For example, the machine learning model may include a deep learning model. The machine learning model may be configured to determine one or more metrics indicative of the health and quality of the sperm sample as discussed above, including but not limited to, concentration of the sperm sample, motility of the sperm sample, and the morphology of at least one sperm of the sperm sample. The machine learning model may, additionally or alternatively, be configured to determine the motion of at least one sperm in the sperm sample along either or both of a 2D path or 3D path. In some embodiments, the machine learning model may be a first machine learning model configured to determine a first subset of the metrics, for example, concentration and motility. In some embodiments, the system may further include a second machine learning model configured to determine a second subset of metrics, for example, morphology of the sperm sample.

The machine learning model may output an indication of the health and quality of the sperm sample based at least on the one or more determined metrics and additionally or alternatively, output the results of the one or more determined metrics themselves. For example, the diagnostic system may be operatively coupled to a display or another device comprising a display and may display the indication of the health and quality of the sperm sample and the results of the one or more determined metrics using the display. In some examples, the display may be part of another computing device like a personal computer, laptop, or a mobile device. In some examples, the machine learning model may include more than one machine learning model. For example, one machine learning model may be configured to determine the concentration of the sperm sample while another machine learning model may be configured to determine the motility of the sperm sample.

In some embodiments, the diagnostic system may be provided as an integrated diagnostic device. For example, the imaging device, processor, and machine learning model may be integrated on the same device. In other examples, the imaging device may be operative coupled with a separate computing device having the processor and machine learning model. In some examples, the processor may further be configured to implement the machine learning model rather than having a separate component for the machine learning model. Although embodiments are described with respect to the above configurations, these are for exemplary purposes only and the diagnostic system may be implemented as any suitable configuration of the combination of the imaging device, processor, and machine learning model.

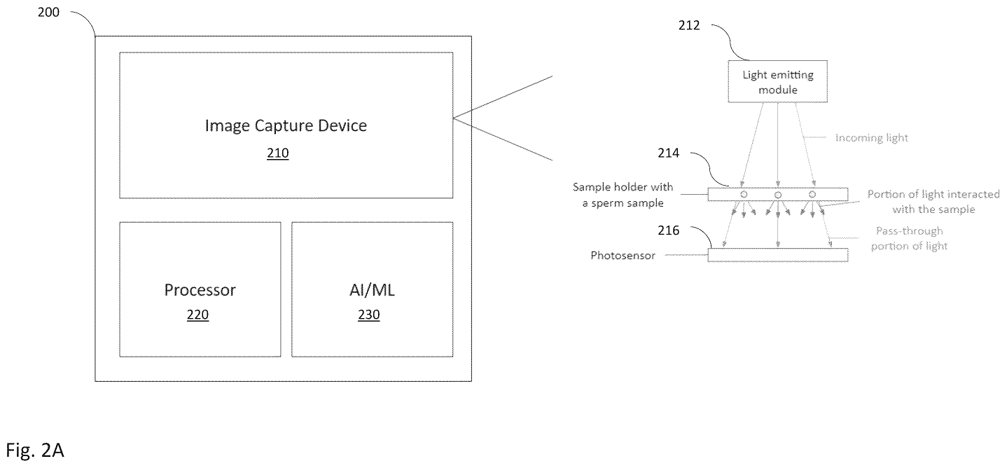

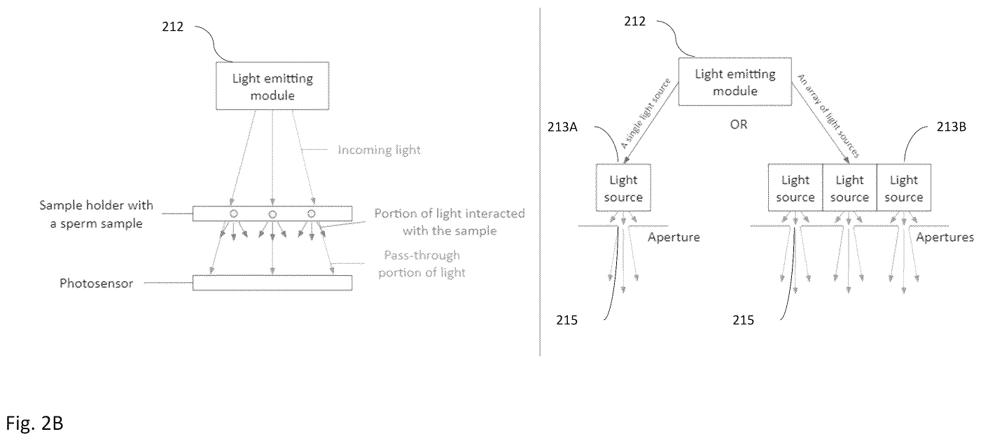

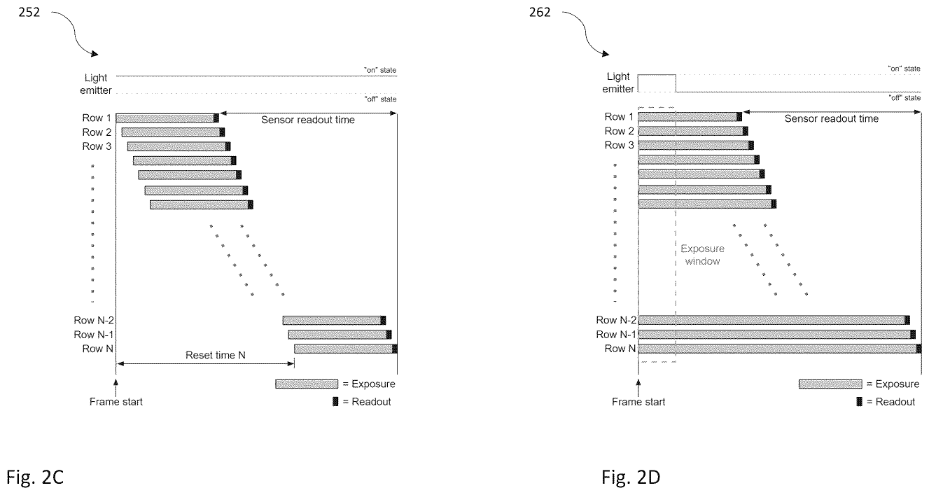

FIG. 2A depicts an exemplary embodiment of a diagnostic system 200 for automated sperm analysis, according to some embodiments. In some embodiments, the diagnostic system may include an imaging device 210, a processor 220, and a model 230 as described above and further herein. FIG. 2A depicts the diagnostic system 200 as integrated on one unitary device, although this is for exemplary purposes only and the diagnostic system 200 may include any combination of devices having the above-indicated components. For example, the imaging device 210 may be operatively coupled with an external computing device (e.g., personal computer, laptop, mobile device, server, etc.) implementing the processor and the model. In some embodiments, the imaging device 210 may be an inline holographic microscopy device or any other suitable imaging device having a light emitting module, sample holder, and sensor. Details of an inline holographic microscopy device will be provided further herein.

FIG. 2B depicts various exemplary configurations of an imaging device 210 for a diagnostic system 200 for automated sperm analysis, according to some embodiments. For example, the light emitting module 212 may comprise a single light source 213A or an array of multiple light sources 213B. In some embodiments, the imaging device 210 may further include one or more apertures or spatial filters 215 associated with the one or more light sources. In some embodiments, the apertures or spatial filters 215 may be integrated with the light emitting module 212 or may be separate from the light emitting module 212.

### Inline Holographic Microscopy

In some embodiments, the imaging device 210 of the diagnostic system 200 may include an inline holographic microscopy device. Inline holographic microscopy devices may provide lower-cost, portable functionality than a typical microscope that requires more components and bulkier components. The inline holographic microscopy device may include an emitter 212 configured to provide light source 213 for illuminating the sample. In some embodiments, the emitter 212 may illuminate the sample through an aperture or spatial filter 215 disposed between the emitter and the sperm sample. In some embodiments, the light source 213 may produce partially coherent light. In some embodiments, the emitter 212 may be a single light source, for example, a light emitting diode (LED), a laser diode, or other type of light source. In some embodiments, the emitter may include multiple light sources, for example, multiple LEDs or laser diodes arranged in an array or arranged in any suitable manner.

The inline holographic microscopy device may further include a sample holder 214 to hold the sperm sample and a sensor 216 to detect the optical signals encoded with information associated with the sperm sample. In some embodiments, the sample holder 214 may allow light to pass through the sample holder 214 to the sensor 216. For example, the sample holder 214 may be disposed between the emitter 212 and the sensor 216. In that way, the sensor 216 can receive the light that passes through the sperm sample holder.

In some embodiments, the sensor 216 may be configured to detect the intensity of the optical signals passing through the sperm sample. In some embodiments, the sensor 216 may be any suitable sensor capable of detecting optical signals and may comprise an optical matrix sensor, for example, a carbon metal oxide semiconductor (CMOS) optical sensor. The sensor 216 may be operatively coupled to the processor (e.g., processor 220) of the diagnostic system to send the detected optical signals to the processor for further processing as described above and further herein.

The sensor 216 may be configured to capture the optical signals in any suitable manner. For example, in some embodiments, the sensor 216 may be configured to operate in an electronic rolling shutter (ERS) scheme. FIG. 2C depicts a graphical representation 252 of an electronic rolling shutter (ERS) image acquisition scheme, according to some embodiments. In the ERS scheme, the emitter 212 may be set to a continuous wave operation mode, where the emitter is on for the entire acquisition window. Each row of the sample may be exposed and each row of the image matrix may be acquired with some time delay before being transmitted to the processor for further processing. The ERS mode may be beneficial for capturing static objects.

Alternatively, the sensor 216 may be configured to capture the optical signals in a global reset release (GRR) scheme. FIG. 2D depicts a graphical representation 262 of a global reset release (GRR) image acquisition scheme, according to some embodiments. In the GRR scheme, the emitter 212 may be set to a pulse mode. As such, the sensor 216 and the emitter 212 may be operatively coupled so as to synchronize frame acquisition with the light pulse. For example, the emitter 212 may be controlled by receiving a signal from the sensor 216. In that way, the emitter 212 and the sensor 216's frame acquisition may be synchronized. Alternatively or additionally, the emitter 212 and/or sensor 216 may be independently controlled. For example, either or both components may be controlled independently by a computing module (of the system or disposed separately). By following the GRR scheme, moving objects may be captured with higher clarity than the ERS scheme since the effective exposure of all rows happens simultaneously when the emitter is pulsed in the “on” state. Further, readout occurs when the emitter is off and no additional information is added during the readout time. In some embodiments, the system may be configured to allow selective switching between the ERS and GRR modes to perform different analyses and device diagnostic checks.

However, the technology may use any suitable image acquisition scheme. The emitter may operate in either or both (e.g., via a switch) the continuous wave or pulse configuration. Further the emitter and the sensor may be operatively coupled and controlled together, or may be controlled separately (e.g., via a compute module). In some embodiments, the emitter may be controlled by receiving one or more signals directly from the sensor. In that way, the components of the imaging device may be configured to operate using any suitable image acquisition scheme.

In some embodiments, the optical signals may be transmitted by the sensor 216 to the processor 220 for further processing, for example, as discussed above to generate a plurality of images of the sperm sample. The detected optical signals may include information about amplitude of the optical signal at the sensor, for example, by relating a detected intensity of the optical signals to the amplitude of the optical signal using a square modulus relationship. It can be appreciated that the detected intensity of the optical signals may include the intensity of the optical signal at the sensor plane rather than at the sperm sample plane. Accordingly, the amplitude and phase components of the optical signals at the sample may be reconstructed or otherwise determined using the detected intensity of the optical signals at the sensor.

To determine the amplitude and phase components of a complex representation of the optical signals at a specific depth, the processor may perform an iterative algorithm. In some embodiments, a machine learning model may be used to perform the iterative algorithm described herein or a suitable reconstruction method to determine the amplitude and phase components. In some embodiments, the iterative algorithm may begin by determining a field of complex representations of the optical signals at a plane of the sensor used to detect the optical signals including initial amplitude and phase values. In some embodiments, the initial phase value may be set to 0 as the sensor may not detect any initial phase information of the optical signals. Then the field may be back-propagated by a distance z representing the optical signal at a first depth of the sperm sample. The back-propagation may determine an initial value for the phase and amplitude components of the complex representations of the optical signal at the specific depth by propagating that result forward back to the plane of the sensor. In some embodiments, the processor may perform only one iteration of the back-and forward-propagation. In other embodiments, the processor may perform many different iterations of propagating the complex field back and forth to refine the amplitude and phase values. For example, on each cycle of back-and forward-propagation, the amplitude and phase values may be iteratively updated based on the results of the propagation cycle. This iterative algorithm may be repeated for various different depths of the sperm sample. For example, the iterative algorithm may back-propagate the complex field to a different value of z (e.g., z₁, z₂, z₃, etc.) each representing different depths of the sperm sample. In that way, the amplitude and phase values of the complex field at the plane of the sensor and at the different depths of the sperm sample may be refined for various depths throughout the sperm sample.

It can be appreciated that a sensor may be limited in the quantity and quality of data that it may be able to detect, and the detection of the optical signals may be limited to the particular resolution and pixel size of a given detector. As such, in some embodiments, instead of a single light source, the emitter of the imaging device may include an electrically controllable light source array, for example, an LED array, or a laser diode array. Accordingly, the electrically controllable light source array may be capable of providing variable illumination conditions (e.g., turning on one LED of an LED array versus turning on a different LED of the LED array). In some embodiments, the illumination condition may include a location of the light source, for example, turning on a first LED in a first position of an LED array versus turning on a second LED in a second position of the LED array. It can be recognized that the illumination conditions of the light source may vary the detected optical signals that pass through the sperm sample. Thus, by detecting various sets of optical signals using different illumination conditions of the light source, the diagnostic device may be capable of reconstructing images of the sperm sample at a higher resolution than the sensor may be able to detect.

In some embodiments, a first set of optical signals may be detected by the sensor using a light source at a first illumination condition and a second set of optical signals may be detected by the sensor using a light source at a second illumination condition. The first set and second set of optical signals may both be associated with the sperm sample at the same point in time. The images of the sperm sample may then be reconstructed using both sets of optical signals. For example, the processor may combine the first and second sets of optical signals to form a higher resolution representation of the sperm sample. Then a plurality of high-resolution images may be generated from the combined first and second sets of optical signals in a similar manner as described above. In some embodiments, the first and second sets of optical signals may be combined by matching features of the first and second sets of optical signals. In some embodiments, an optimization calculation may be performed on the combined first and second sets of optical signals to optimize the resultant high-resolution images. These higher resolution images may then go through a similar phase recovery method as described above. Although only two sets of optical signals are used in this description, it can be appreciated that any number of sets of optical signals may be detected at various illumination conditions and used to generate the high-resolution images.

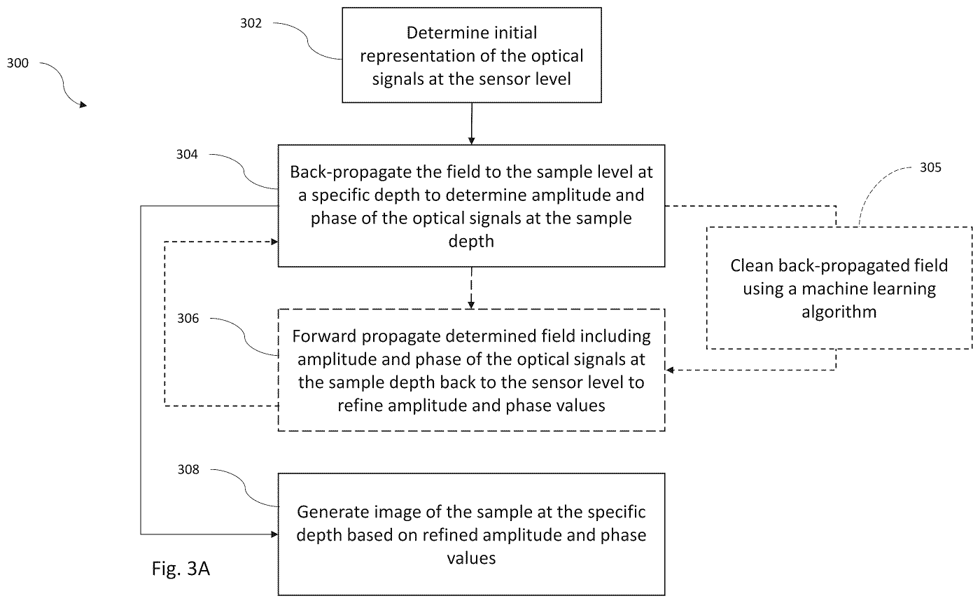

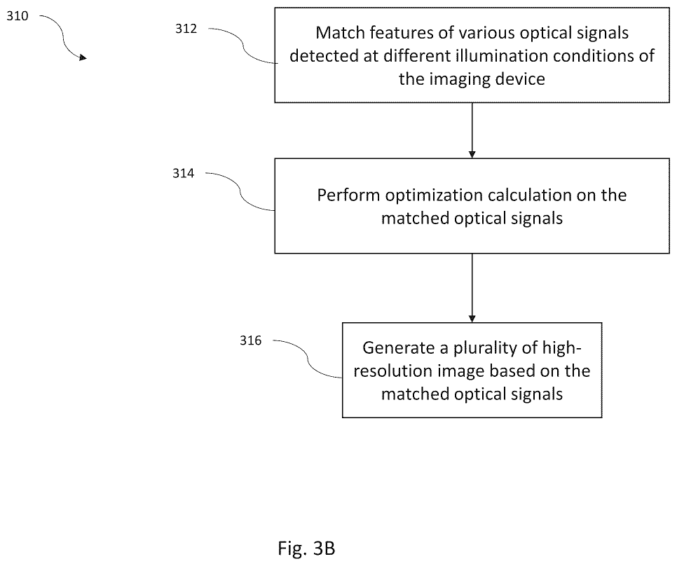

FIG. 3A depicts an exemplary computational flow 300 for reconstructing an image of a sperm or other sample captured by an imaging device, according to some embodiments. The flow may start, at step 302, by determining an initial representation of the optical signals at the sensor level. For example, the representation may be determined to have an amplitude value based on the detected intensity of the optical signals as discussed above, whereas a phase value of the representation may initially be set to 0.

At step 304, the flow may then back-propagate the field from the sensor level to the sample level at a specific depth to determine an updated amplitude and phase value of the optical signals at the sample depth. In some embodiments, after a single back-propagation, the flow may proceed directly to step 308 to generate the images of the sample.

Optionally, the flow may iterate and may first forward propagate the field determined at step 304. As such, at step 306, the determined amplitude and phase value representation at the sample depth may then be forward propagated to further refine the amplitude and phase values. Optionally, these steps may be repeated to further refine the amplitude and phase values of the optical signals at the sample depth.

At step 308, the flow may then generate an image of the sample at the specific depth based on the refined amplitude and phase values.

Additionally or alternatively, in some embodiments, the processor may execute a machine learning algorithm, at step 305, to perform the iterative algorithm. The machine learning algorithm may be trained to more accurately back-and forward-propagate the values to provide more efficient reconstruction. In some embodiments, the machine learning model may be pre-trained to clean the image generated as described above. For example, in-line holographic techniques may be prone to twin-image artifacts, which refer to features of the original object (e.g., the sample) and its inversion appearing simultaneously thereby affecting the accuracy of the reconstructed image. In some embodiments, the machine learning model may be trained to filter out noise artifacts such as twin-image artifacts, or any other artifacts that may arise during the reconstruction process. The machine learning model may remove twin-image artifacts from the back-propagated field prior to the forward propagation step. Then, the “clean” field may be forward propagated as described above to the sensor plane. At the sensor plane, the resulting amplitude may be replaced. In some embodiments, the resulting amplitude may be replaced with a measured amplitude value based on the detected intensity. In some embodiments, the measured amplitude used in the replacement is the square root of the detected intensity value. By using a machine learning algorithm, the algorithm may only utilize one iteration of back- and forward-propagation to reconstruct the image of the sample.

Any suitable machine learning algorithm may be used, for example, a neural network, deep-learning neural network, convolutional neural network, or any other suitable machine learning algorithm. In some embodiments, multiple machine learning models may be used, for example, a first machine learning model may be used for the amplitude values of the generated optical field and a second machine learning model may be used for the phase values of the generated optical field.

Any of the techniques described above with respect to FIG. 3A may be used in combination with each other or independently. For example, a physical solver may be used to perform direct propagation (e.g., a single set of back and forward propagation) of the measured components or may perform the iterative approach described above. Additionally or alternatively, a machine learning model may be trained and used to perform the reconstruction and cleaning described above.

FIG. 3B depicts an exemplary computation flow 310 for reconstructing a high-resolution image of a sperm or other sample based on various detected optical signals of the sample, according to some embodiments. At step 312, the flow begins by having the processor may match various optical signals detected using different illumination conditions of the imaging device. For example, the imaging device may detect a first set of optical signals at a first illumination condition and a second set of optical signals at a second illumination condition, although it can be appreciated that any number of sets of optical signals may be detected by the imaging device for use in the method. The first and second sets of optical signals may then be combined by matching the features. In some embodiments, at step 314, the processor may then perform an optimization calculation on the matched optical signals to optimize the combination of the sets of optical signals. At step 316, a plurality of high-resolution images may be generated based on the matched optical signals according to any suitable method, for example, the method described herein with respect to FIG. 3A. In some embodiments, the higher resolution image may represent a sub-pixel resolution image where the pixel size of the sub-pixel resolution image is smaller than the pixel size that the sensor of the imaging device is able to detect.

### Deep Learning Algorithms for Sperm Health Analysis

It can be appreciated that a number of different imaging devices may be available to detect the optical signals. It can be further appreciated that the various imaging devices may have different physical limitations as discussed above and further herein, for example, different resolutions, different detection thresholds, etc. In some embodiments, an imaging device with a lower resolution may be preferable, for example, for accessibility to point-of-care sperm analysis facilitated by physically smaller or more adaptable devices. Accordingly, in some embodiments, the machine learning model may be configured to accurately analyze images at a variety of resolutions lower than a preferred resolution. The machine learning model may provide more stability for the model across datasets with various distributions and may provide more accurate image classification for low-resolution images. In some embodiments, the machine learning algorithm may be a deep learning algorithm configured to determine an indication of the health or quality of the sample.

In some non-limiting embodiments, the machine learning model may be a deep learning object-detection model or a deep learning image classifier model, or any other suitable deep learning model. The deep learning model may be configured to determine at least a subset of the metrics indicative of the health or quality of the sperm sample, for example, concentration and motility of the sperm sample. The deep learning model may be configured to detect sperm cells among the various pixels of the received images by training the deep learning model on labeled training images. The labeled training images may include both high resolution and low-resolution images or may include a spectrum of images with various resolutions. In some embodiments, the deep learning model may be trained using training images of the same resolution as the resolution of the sensor. The deep learning model may be configured to further count the number of detected sperm cells within the image and determine a concentration of the sperm sample based at least in part on the counted number. The deep learning model may further use a dilution measure and/or volume measure of the sperm sample to determine the concentration of the sample. Additionally or alternatively, the deep learning model may be configured to detect an orientation and location of each sperm cell in the sperm sample based on the received figures. The deep learning model may compare the detected orientation and location of a sperm cell in an image representing a first frame of the sperm sample as compared to the detected orientation and location of the sperm cell in an image representing a second frame of the sperm sample or multiple images representing multiple later frames of the sperm sample to determine motility and motion tracking of the sperm sample.

In some non-limiting embodiments, the machine learning model may be an adversarial neural network. An adversarial neural network may provide more stability for the neural network across datasets with various distributions and may provide more accurate image classification for both lower and high-resolution images that may occur when capturing the images of the sperm sample. Adversarial neural networks may also provide benefits over other machine learning models that may, for example, require more supervised learning during the training of the machine learning model and may ultimately be limited to the distribution of the training data. For example, a machine learning model that follows supervised learning using a dataset obtained from one type of imaging device may not perform well when analyzing data obtained from a different type of imaging device or exhibit a decrease in performance when the target data is at a lower resolution than the training data. Adversarial neural networks may provide the adaptability to maintain high accuracy results when dealing with images and data from various sources that may have different parameters such as field-of-view, magnification, resolution, or other various parameters.

In some embodiments, the machine learning model may be an adversarial neural network having a first portion of the model and a second portion of the model. The adversarial neural network may be configured to determine an indication of the health and quality of the sperm sample, including for example, a morphology assessment as described above and further herein. As discussed above, the adversarial neural network may receive the generated images from the processor of the diagnostic system. The first portion of the model of the adversarial neural network may be configured to generate data indicative of the health and quality of the sperm sample. For example, the first portion of the model may be an image classifier model configured to determine whether a sperm cell in the sperm sample is a normal or abnormal shape. The image classifier may classify the images by using a feature extractor and determine the classification based on the features extracted by the feature extractor. The second portion of the model may be configured to evaluate the data generated by the first model and refine the data generated by the first model. For example, in assessing morphology, the second portion of the model may be configured to determine if a shape of the sperm in the sperm sample is a normal or abnormal shape based at least in part on the results of the first portion of the model. In some embodiments, the second portion of the model may analyze the features extracted by the first portion of the model and determine which features are recurrent in all domains (e.g., a source domain having high resolution images, and a target domain having low resolution images). In this way, the adversarial neural network may be made domain independent and may accurately differentiate between the different domain distributions. For example, the adversarial neural network may be trained on a source domain of training data having high-resolution images but may be used to analyze data in a target domain of data having lower resolution images than the training data of the source domain. The output of the model may then be determined using the data generated by the first portion of the model and the refined data generated by the second portion of the model. For example, both the data generated by the first portion and the refined data generated by the second portion of the model may be used as input to an optimization process to determine whether the sperm is a normal or abnormal shape. In some embodiments, rather than the first portion performing both feature extraction and classification, the adversarial neural network may additionally or alternatively include a third portion of the model to determine the features of the plurality of images to be used by the first and second portions of the model to generate the data indicative of the health and quality of the sperm sample.

In some embodiments, the adversarial model may be trained on data from a source domain. The source domain may include higher resolution data, data labeled by specially trained professionals indicating that the data depicts certain metrics, or data that is both higher resolution and labeled. In some embodiments, the source data may be obtained from a first imaging device, for example, a tabletop microscope that produces higher quality images of sperm samples. In some embodiments, the adversarial neural network may further be trained on a target domain which may include data that is lower resolution than the source domain data or may be unlabeled or may be both unlabeled and lower resolution. In some embodiments, training of the adversarial neural network may include using a feature extractor of the adversarial neural network to extract different feature representations, for example, to distinguish a sperm cell of the sperm sample, in data from both the source and target domains. The extracted features may be used by the first and second portions of the model models to accurately differentiate between the different domain distributions. This training method is described for exemplary purposes only, and any suitable method to train the model may be used.

Although the above features are described in the context of an adversarial neural network, it can be appreciated that any suitable machine learning model may be used in place of the adversarial neural network for analyzing lower-resolution images than what the model was trained on. For example, the machine learning model may be configured to adapt to lower-resolution input images following a variety of domain adaptation strategies including, but not limited to, adversarial discriminative domain adaptation, domain-adversarial neural networks, deep adaptation networks, pixel-level domain adaptation, conditional domain adaptation networks, generative adversarial guided learning, contrastive adaptation networks, or any other suitable domain adaptation strategy or combination thereof.

It can be further appreciated that the machine learning model of the diagnostic system may be a first machine learning model and the diagnostic system may include more than one machine learning model. In some embodiments, the diagnostic system may have a first machine learning model that may be a deep learning model configured to determine concentration and motility of the sperm sample, as described above, and a second machine learning model configured to determine morphology of the sperm sample as described above. For example, the first machine learning model may be a deep learning image classifier model configured to detect the sperm cells in the sperm sample to determine concentration and motility and the second machine learning model may be an adversarial neural network or any other suitable model configured to determine the morphology of the sperm cells in the sperm sample.

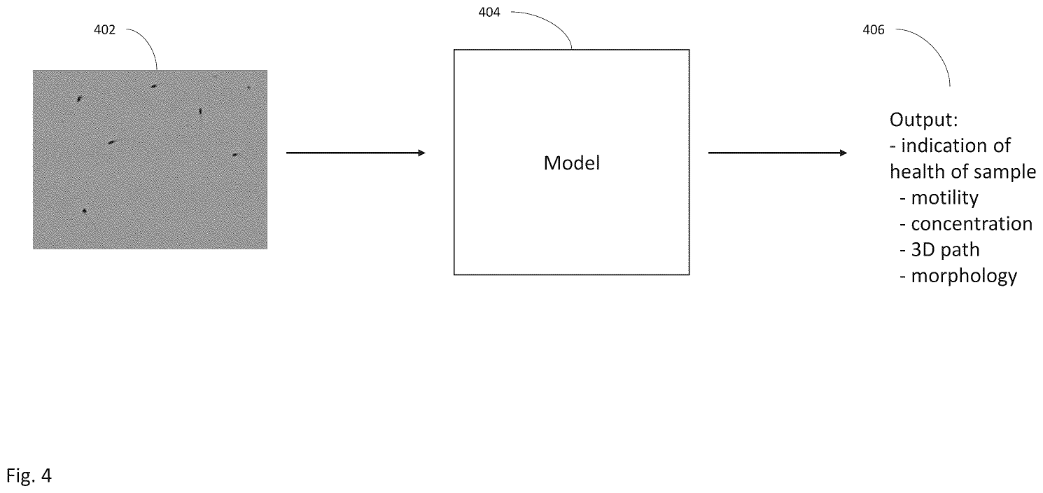

FIG. 4 depicts an exemplary machine learning model for determining an indication of the health or quality of a sperm sample based at least in part on received 2D or 3D images, according to some embodiments. The model may receive one or more of the images 402 generated by the processor for use in determining an indication 406 of the health or quality of a sperm sample based at least in part on the received images. The images may be received in any suitable manner from the processor, for example, over a wired or wireless connection. In some embodiments, the model may 404 be an adversarial neural network as described above.

### Diagnostic Metrics and Model Configurations

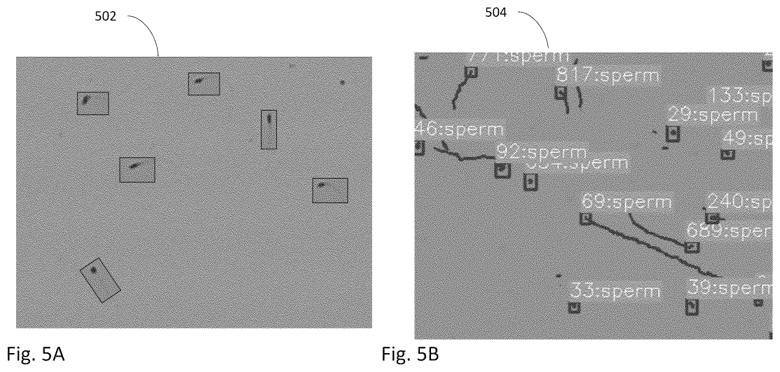

As discussed above, various metrics indicating the health and quality of the sperm sample may be determined, including but not limited to, concentration of the sperm sample, motility of the sperm sample, and morphology of the sperm sample. In some embodiments, one or more of the metrics may be determined using a machine learning model as described herein. In some embodiments, one or more metrics may be determined using conventional processing while other metrics are determined using a machine learning model. In some embodiments, multiple machine learning models may be used. For example, motility and concentration may be determined using an object-detection deep learning model or any other suitable model where morphology is determined using an adversarial neural network. FIGS. 5A-5B depict exemplary outputs of the model including metrics indicating the health or quality of a sperm sample, according to some embodiments. FIG. 5A depicts an output 502 of the concentration of the sperm sample. In some embodiments, the component configured to determine the metrics may first detect the sperm cells within the images generated by the processor. The component may then determine the number of sperm cells within the frame of the image or the entire sample and use that number to determine the concentration of the sperm sample. In some embodiments, the frame of the image may not capture the entire sperm sample and may use other parameters including, but not limited to, total volume and dilution to extrapolate the total concentration. FIG. 5B depicts an output 504 of the motility of the sperm sample. In some embodiments, the component configured to determine the metrics may first detect the sperm cells within the images generated by the processor associated with a first point in time. The component configured to determine the metrics may then detect whether the sperm cells have moved positions by detecting the sperm cells within the images generated by the processor associated with a second point in time later than the first point in time and comparing the position of the sperm cells in the two sets of images.

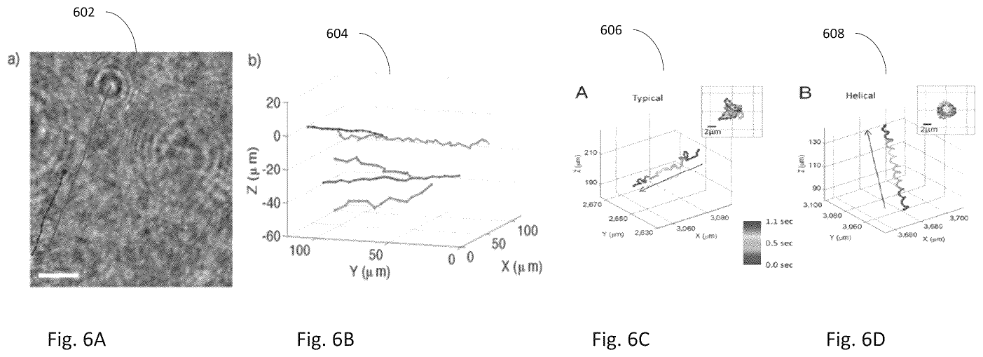

The component configured to determine the metrics indicative of the health and quality of the sperm sample may further determine the path of motion of one or more sperm cells within the sperm sample. The determined path of motion may be a 2D projection of the sperm cell's path or may be a 3D path of the sperm cell through the sperm sample. FIGS. 6A-6D show examples as depicted in “High-Throughput Lensfree 3D Tracking of Human Sperms reveals rare Statistics of Helical Trajectories” (Su et al., 2012) (hereinafter 3D Tracking) which is hereby incorporated by reference, which depict exemplary outputs of a model including path tracking of a sperm sample, which can be used to analyze a sperm sample according to some embodiments. FIG. 6A depicts a 2D projection 602 of the path of various sperm cells within the sperm sample. The 2D projection may alternatively or additionally be displayed in a graphical format and may be shown from any perspective as depicted in FIGS. 6C and 6D. FIG. 6B depicts the related 3D paths of the various sperm cells within the sperm sample as shown graphically. For example, the output of the diagnostic device may include a 3-axis graph 604 showing the path of the sperm cells. In some embodiments, the 3D paths may be shown in a reconstructed 3D representation of the sperm sample in addition or alternatively to the graphical representation.

In some embodiments, the component configured to determine the metrics indicative of the health and quality of the sperm sample may determine a classification or label for the path of the various sperm cells within the sperm sample. FIG. 6C depicts a 3D graph 606 showing the path of a sperm cell within the sperm sample. In some embodiments, the component configured to determine the metrics may further determine where the sperm cell is at a given point and provide an indication (e.g., coloring the path) of where the sperm cell is at a given point. For example, the 3D graph may include a colored path indicating the point in time. A first color may indicate the starting point of the diagnostic detection at t=0 seconds. The color may change as time passes through t=0.1 s, 0.2 s, 0.3 s, etc. after the starting point and the color of the path may reflect this passage of time. This information may be further used to determine other metrics like speed of the sperm cell.

The component configured to determine the metrics may determine based on the reconstructed or tracked path, that the sperm is moving normally or not. FIG. 6C depicts a 3D graph 606 showing the path of a sperm cell within the sperm sample moving in a typical path. FIG. 6D depicts a 3D graph 608 showing the path of a sperm cell within the sperm sample moving in a helical path. While typical and helical paths are depicted, this is for exemplary purposes only and other path indicators, for example, hyper-activated, hyper-helical, or any other suitable path indicator may be determined and displayed.

### Other Biological and Non-Biological Use Cases

As discussed above, the systems and techniques described above with respect to automated sperm analysis, can be adapted to suit other biological and non-biological analyses. The systems and techniques described herein may be used for any suitable analysis, including but not limited to: automated sperm analysis, urinalysis, hematology analysis, microbiological and cell biology analyses, biological fluid analyses (cerebrospinal, thoracentesis, pleural fluid, joint fluid, etc.), and biopsy analysis (mass, urological, prenatal, etc.), the details of which will be described further herein. In some embodiments, the systems and techniques may be adapted to suit another type of analysis with minimal alterations.

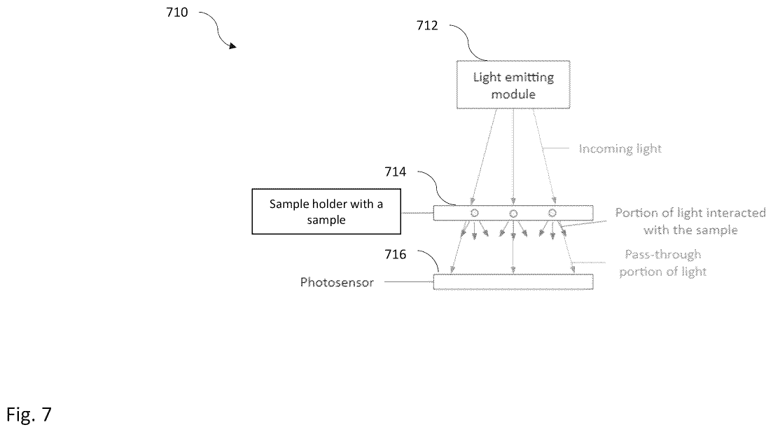

FIG. 7 depicts an exemplary configuration of an imaging device 710 for a diagnostic system for automated sample analysis, according to some embodiments. The exemplary imaging device may include a light emitting module 712, a sample holder 714, and a photosensor 716. The components of the imaging device may be configured in numerous ways. For example, the light emitting module 712 may comprise a single light source or an array of multiple light sources. In some embodiments, the imaging device 710 may further include one or more apertures or spatial filters associated with the one or more light sources. In some embodiments, the apertures or spatial filters may be integrated with the light emitting module or may be separate from the light emitting module.

As described above with respect to the sperm analysis, the imaging device 710 may be an inline holographic microscopy device configured to detect optical signals encoded with information regarding the sample. The inline holographic microscopy device may be substantially similar for other analyses described herein as was described above with respect to the sperm analysis (e.g., imaging device 212). For example, the inline holographic microscopy device may include a light emitting module 712 having one or more light sources to illuminate the sample, a sample holder 714 configured to hold the sample to be analyzed, and a sensor 716 (e.g., a photosensor) to detect the optical signals encoded with information associated with the sample for further analysis. However, one or more optical or mechanical properties of the device may be adjusted to account for the differences in sample types.

For example, the inline holographic microscopy device may include a light source for illuminating the sample as described above. However, it can be appreciated that different types of samples may benefit from being illuminated by different wavelengths of light. As such, in some embodiments, the light source may produce a particular wavelength or particular range or wavelengths suited for the sample type. Further, rather than having to switch out a light source, in some embodiments, the light emitting module may include a light source configured to emit white light and a filter and/or aperture configured to allow a particular wavelength or range of wavelengths to pass through to illuminate the sample. The particular wavelength or range of wavelengths may be suited for a particular sample type.

Further, each sample type may have different physical properties, such as viscosity, or state of matter (e.g., liquid versus powdered samples), that may be suited to be held by a particular type of sample holder so that the sample may be properly illuminated in the imaging device. Any suitable sample holder may be used, for example, a slide, sample well, cuvette holder, or any other suitable sample holder. Each type of sample holder may have different optical or mechanical properties. For example, a thin slide sample holder may have a small thickness and substantially uniform optomechanical properties of a sample region. However, a sample well may have a recessed portion for holding a less viscous sample, which may have a greater thickness than a thin slide and may be shaped in a way that affects how the light may interact with the sample or pass through the sample holder. As such, the imaging device may be adapted to account for the different optomechanical properties of the sample holder to be used. For example, in some embodiments, a distance between the light emitting module and the sensor may be increased to allow for a thicker sample holder to be placed in between. In some embodiments, for example in embodiments with multiple light sources, the arrangement and geometry of the light source(s) may be adjusted. In some embodiments, rather than adjusting the imaging device itself, the difference in optomechanical properties may be addressed at the processing stage through a normalization process.

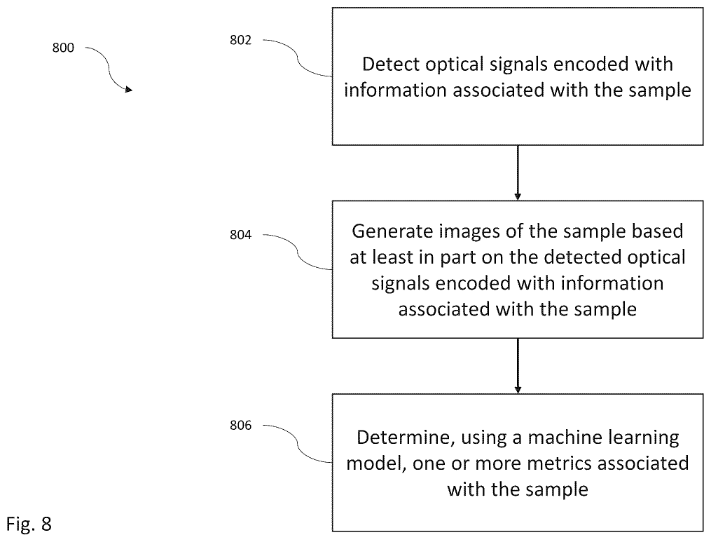

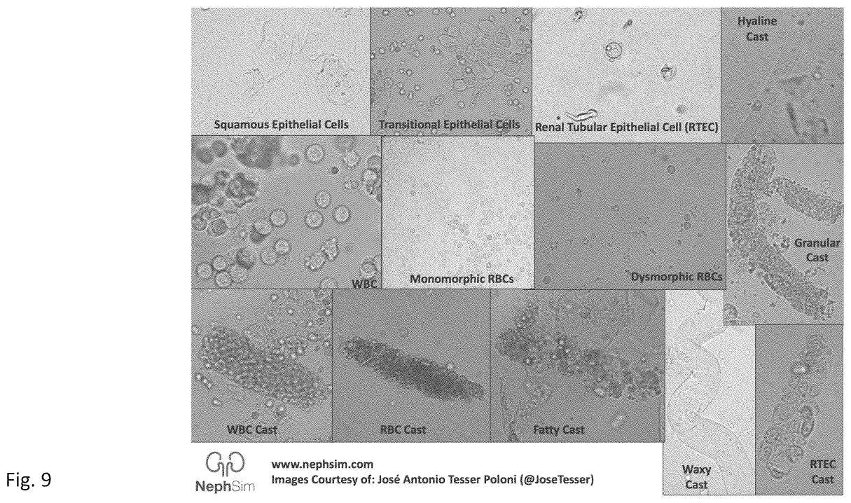

FIG. 8 depicts an exemplary method 800 for automated sample analysis, according to some embodiments. The method may be substantially similar to the method for automated sperm analysis described with respect to FIG. 1. For example, the exemplary method may begin, at step 802, by detecting optical signals encoded with information associated with the sample. As with the sperm analysis, when light passes through the sample, some of the light may interact with the sample and some may pass through the sample without any interaction, both of which may be indicative of aspects of the sample, including depth, thickness, or any other suitable characteristic. The encoded information may be detected by detecting the light that passes through the sample with and without interaction.

At step 804, the method may then proceed to generate one or more images of the sample based at least in part on the detected optical signals encoded with information associated with the sample. As described above with respect to FIG. 3A, the optical signals detected by the sensor of the imaging device may provide an initial representation of the optical signals at the sensor level. This initial representation can be used to initialize the iterative back-and forward-propagation method described in FIG. 3A. Further, in embodiments with more than one light source, the sample may be illuminated under more than one illumination condition to generate higher-resolution images of the sample. As described with respect to FIG. 3B, the optical signals detected under each different illumination condition may be combined by performing a feature matching process and the combined optical signals may be optimized prior to generating the higher-resolution images. In that way, sub-pixel resolution of the generated images may be achieved.

At step 806, the generated images may then be analyzed to determine one or more metrics associated with the sample. While different metrics may be suited for different analyses, the one or more metrics may generally include: the presence of a specific inclusion (e.g., molecule, cell, microorganism, etc.) within the sample, the prevalence of a specific inclusion within the sample, the morphology of one or more inclusions within the sample, the color of the sample, or any other suitable metric for analyzing the sample. FIG. 9 depicts images of one or more inclusions that may be evaluated and identified in a sample using the systems and techniques described herein, according to some embodiments.

The analysis may be performed by a machine learning model executed by one or more processors (CPU, GPU, NPU, etc.) as described herein, and may include a deep learning model, adversarial neural network, image classifier, or any other suitable machine learning model as described herein. While many aspects of the machine learning model may be the same or substantially similar to the machine learning models described above with respect to automated sperm analysis, the machine learning models used for the various analyses described further herein may be trained to determine one or more attributes regarding the sample. For example, the machine learning model may recognize different features suited for the particular analysis including, but not limited to, the presence of an inclusion in the sample, the size, shape, or movement of a particular inclusion, the number of a particular inclusion (e.g., concentration, number per imaged sample area, number per high or low powered field). Details of the attributes and training of the machine learning model for a non-limiting group of potential analyses are described further below.

### Urinalysis

A conventional urine analysis typically consists of three parts including: (1) a visual inspection, (2) a dipstick for chemistry analysis, and (3) a microscopic evaluation to determine the different inclusions in the sample. In some embodiments, the systems and techniques described herein may be configured to perform a microscopic evaluation of a urine sample by identifying the presence, type, and quantity of one or more inclusions in the sperm sample. The systems and machine learning techniques described herein may provide faster and more accurate microscopic analyses of a urine sample than would be performed by a laboratory technician or other personnel, or by traditional computational techniques. As a urinalysis may be performed for many different inclusions (e.g., cells, organisms, etc.) of interest, a machine learning model, or machine learning models, may be trained to analyze the urine sample to identify metrics associated with one particular inclusion, one type of inclusion, or a group of inclusions. The microscopic evaluation may be performed to evaluate one or more metrics associated with inclusions in the urine sample such as, but not limited to casts, cells, microbes/microorganisms, and/or urinary crystals.

In some embodiments, the machine learning model may be configured to identify and evaluate metrics associated with one or more types of casts found in a urine sample. Casts may comprise tube-like structures approximately 10-15 micrometers in size and composed of cells and proteins that may originate from a distal convoluted tubule and/or the collecting ducts of a kidney. The presence of casts in a urine sample may be indicative of intrinsic kidney diseases including, but not limited to, nephrotic syndromes, nephritic syndromes, kidney infections, acute tubular injury, acute tubular necrosis. As seen in FIG. 9 a number of different types of casts, including red blood cell casts (RBC casts), white blood cell casts (WBC casts), epithelial cell casts, granular casts, hyaline casts, waxy casts, and/or fatty casts, each of which may be indicative of a particular intrinsic kidney disease or multiple potential intrinsic kidney diseases. For example, the presence of red blood cell casts in urine may signify bleeding in a portion of the kidneys or the presence of white blood cell casts in urine may signify a kidney infection.

As such, the machine learning model used to perform the analysis of the generated images by identifying one or more types of casts present in the urine sample and evaluate one or more metrics associated with each type of cast identified. For example, the machine learning model may be trained as described herein to determine the tube-like morphology and/or size of a particular type of cast (or multiple types of casts) that may be present in the urine sample.

Alternatively or additionally, in some embodiments, the machine learning model may be configured to identify and evaluate metrics associated with one or more types of cells found in a urine sample. The cells present in a urine sample may include red blood cells, white blood cells (e.g., neutrophils, eosinophils, basophils), and epithelial cells, although other cells may be present as well. The presence of one or more cell types in a urine sample may indicate particular pathologic states, such as a urinary tract infection, inflammation, or urinary tract bleeding. As discussed above with respect to casts, the morphology and size of the various cell types may vary (e.g., cells may range from 6-20 micrometers in size, 1-50 micrometers in size, 6-100 micrometers in size, or any other suitable size).

Alternatively or additionally, in some embodiments, the machine learning model may be configured to identify and evaluate metrics associated with one or more types of microbes or microorganisms found in a urine sample. Microbes and microorganisms, typically around 1-20 micrometers in size, present in a urine sample may be indicative of a urinary tract infection or may indicate that the sample has been contaminated. The presence of microbes in a urine sample may indicate to a laboratory technician or other personnel that a urine culture should be performed to determine the number of colony forming units.

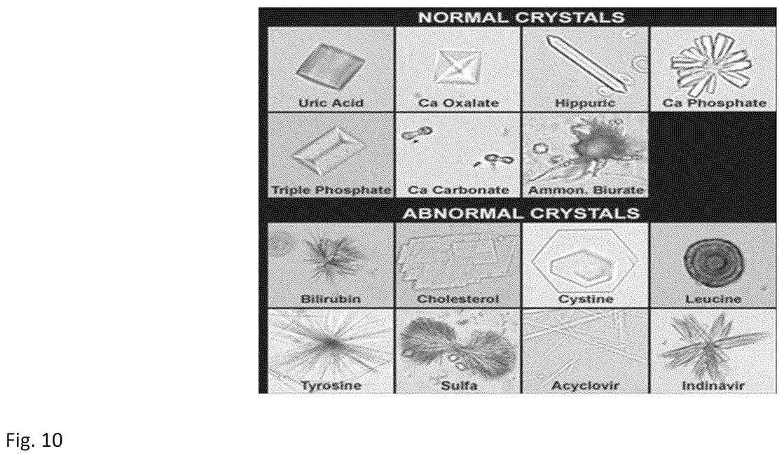

Alternatively or additionally, in some embodiments, the machine learning model may be configured to identify and evaluate metrics associated with one or more crystal precipitates present in the urine sample. Certain metabolic outputs precipitate as crystalline structures having different forms and sizes ranging from approximately 2-20 micrometers. FIG. 10 depicts a non-limiting group of crystalline precipitates that may be present in a urine sample, according to some embodiments. Each of the precipitates may be indicative of a particular disease, or may alert a laboratory technician that more testing needs to be done. For example, uric acid may indicate acidic urine or hyperuricosuria; calcium oxalate may indicate hyperoxaluria or ethylene glycol poisoning; calcium and magnesium phosphate may indicate overly alkaline urine, overactive parathyroid hormone or hyperparathyroidism, or may indicate a calcium-rich diet; struvite may indicate overly alkaline urine or a urinary tract infection from urease predicting bacteria; cysteine may indicate cystinuria; and sulfur may indicate that a person is taking antibiotics that contain sulfa. However, the systems and techniques described herein may be used to evaluate other crystalline precipitates other than those listed, as the technology is not limited in that respect.

In some embodiments, a machine learning model may evaluate the number of each type of inclusion identified. For example, a threshold number of a type of inclusion present may be indicative of a disease or pathologic state, rather than just the presence of the inclusion. In some embodiments, the machine learning model may be configured to output a metric indicative of the number of a type of inclusion identified. For example, the machine learning model may be configured to output a metric indicative of the concentration of a particular inclusion (e.g., casts, cells, etc.) For example, the metric may provide the concentration of the particular inclusion as a number of the particular inclusion in the imaged sample area, or any other suitable metric indicating the concentration or amount of the particular inclusion in the sample. In some embodiments, the metric indicative of the concentration may be standardized to provide a standard metric suitable for comparison across samples or between a sample and a reference value. In some embodiments, a single machine learning model or adversarial neural network as described herein may be used to identify and evaluate all types of inclusions described herein. In some embodiments, multiple machine learning models or adversarial neural networks may be used, each of which is trained to identify a particular type of inclusion (e.g., a cast versus a cell) or a particular inclusion (e.g., RBC cast versus epithelial cell cast).

### Hematology

As with the sperm analysis described above, the systems and techniques described herein may be used to perform a hematological analysis on a blood sample. The system may be configured to provide an analysis of one or more metrics of the blood sample to provide details that may indicate certain blood or blood-related diseases or pathologic states.

For example, the machine learning model may be configured to identify one or more inclusions in a blood sample, including but not limited to red blood cells, white blood cells, thrombocytes, blood parasites (e.g., Plasmodium species, babesia, trypanosome, etc.), or other cells and structures like leukemic blasts, fragmented cells, or otherwise. The machine learning model may be configured to identify the one or more inclusions by analyzing and identifying the morphology of components of the blood sample. Further, in analyzing the morphology, the machine learning model may be configured to identify and evaluate the morphology of a specific inclusion. For example, the machine learning model may be configured to analyze and evaluate the morphology of the red blood cells in the blood sample to determine one or more characteristics associated with a proper morphology, or a morphology indicative of a blood disease such as sickle cell anemia. Further, in identifying the morphology of one or more inclusions in the blood sample, the machine learning model may be configured to identify blood parasites that may be present in the blood sample, including but not limited to, plasmodium species which may cause malaria, babesia, trypanosoma, or other parasites or parasitic structures in the blood sample. In some embodiments, the machine learning model may be configured to determine a complete blood count of red and white blood cells, which may provide additional information regarding blood diseases and autoimmune diseases.

### Other Biological Fluid Analyses

In some embodiments, the systems and techniques described herein may be used to evaluate other biological fluids obtained through one or more medical procedures that may be performed in the course of evaluation and diagnosis of a patient. As described above with respect to the urinalysis, the machine learning model in these analyses may be configured and trained to identify and evaluate one or more metrics associated with different inclusions that may be present in the biological fluid sample. For example, the machine learning model may be configured to identify the inclusions by identifying the morphology of the inclusions in the sample and quantify each inclusion by determining the number of each inclusion present in the sample per microliter of sample.

For example, the system and techniques may be used to evaluate pleural fluid obtained during a thoracentesis, which may be useful in pulmonological, surgical, or intensive care applications. Helpful inclusions of interest in a thoracentesis analysis may include one or more of cells, such as red blood cells, white blood cells, mesothelial cells, malignant cells, and/or eosinophils; microbes and microorganisms such as bacteria, fungi, and/or mycobacteria (e.g., Mycobacterium tuberculosis); or other components such as cholesterol crystals or amorphous debris, although the technology is not limited in this respect.

Additionally or alternatively, in some examples, the systems and techniques may be used to evaluate synovial fluid obtained from a joint during an arthrocentesis, which may be useful in orthopedic applications. Helpful inclusions of interest in an arthrocentesis analysis may include one or more of cells, such as red blood cells, white blood cells, and/or synovial lining cells; crystals, such as monosodium urate crystals, calcium pyrophosphate dihydrate crystals, and/or cholesterol crystals; microbes and microorganisms such as bacteria and/or fungi; or other components such as fat droplets and/or amorphous debris and material, although the technology is not limited in this respect.

Additionally or alternatively, in some examples, the systems and techniques may be used to evaluate ascitic fluid obtained from the abdomen during a paracentesis which may be helpful in gastrointestinal, hepatological, surgical, or intensive care applications. Helpful inclusions of interest in a paracentesis analysis may include one or more of cells such as red blood cells, white blood cells, mesothelial cells, and/or malignant cells; microbes and microorganisms such as bacteria, fungi, and/or mycobacteria (e.g., Mycobacterium tuberculosis); or other components such as fat globules and/or amorphous debris and materials, although the technology is not limited in this respect.

Additionally or alternatively, in some examples, the systems and techniques may be used to evaluate cerebrospinal fluid to provide information about infection, inflammatory diseases, hemorrhages, and/or malignancies affecting the central nervous system. Helpful inclusions of interest in evaluating cerebrospinal fluid may include cells such as red blood cells, white blood cells, and/or malignant cells; or microbes and microorganisms such as bacteria, fungi, although the technology is not limited in that respect.

### Biopsy Analyses

In some embodiments, the systems and techniques described herein may be used to evaluate other biological objects obtained through one or more biopsy procedures that may be performed in the course of evaluation and diagnosis of a patient. For example, the system may be configured to perform microscopic examination of cells and tissue fragments from one or more tissue sources such as, thyroid mass cells, breast mass cells, liver mass cells, lung mass cells, kidney mass cells, soft tissue mass cells, gallbladder mass cells, lymph node cells, prostate cells, embryo cells, or any other suitable tissue source. As described above with respect to the other analyses herein, the machine learning model in these examinations may be configured and trained to identify and evaluate one or more metrics associated with different cells and tissues that may be present in the biological sample. For example, the machine learning model may be configured to identify the presence and quality of the sample, identify and evaluate the types of cells in the sample, evaluate the number of cells in the sample, or perform any other suitable analysis.

### Water Quality Assessment and Industrial Applications

It can be appreciated that the systems and methods described herein may not be limited to biological or medical applications, and may be used to perform analyses of samples in water quality assessments or other environmental, industrial, or non-biologic applications. For example, microscopic entities, including bacteria, protozoa, algae, and other microorganisms, play a big role in evaluating the ecological health of bodies of water and the drinkability of water. As such, the systems and techniques described herein may be configured to perform analysis of a water sample to identify and evaluate one or more metrics associated with the microscopic entities present in the water sample.

In some embodiments, the machine learning model may be configured to identify and quantify the various microscopic entities present in a water sample based on images generated with a system as described herein. The microscopic entities may include bacteria, which may range from approximately 0.5-5 micrometers, and which may be an indicator of contamination, for example, coliform bacteria and E. coli. Additionally or alternatively, the microscopic entities may include protozoa, which may range from approximately 10-50 micrometers, and which may be an indicator of contamination or waterborne diseases. Additionally or alternatively, the microscopic entities may include algae, which may range from 2-100 micrometers, and which may be an indicator of nutrient levels and algal blooms in natural water bodies or may indicate water source issues as related to drinkability. Additionally or alternatively, the microscopic entities may include zooplankton, which may range from 200 micrometers to several millimeters, and which may be an indicator of water quality and ecological health. Additionally or alternatively, the microscopic entities may include fungi and yeasts, which may range from 3-20micrometers, and which may be an indicator of water treatment inefficiencies or storage issues in drinking water, as well as potential contamination in ecological assessments. In some embodiments, the machine learning model may identify the microscopic entities based on a morphological assessment of a component in the sample based on the generated images. By identifying the microscopic entities, the machine learning model may be used to identify specific pathogens in the water sample such as Giardia, Cryptosporidium, E. coli, or any other specific pathogen of interest.

In some embodiments, the systems and methods described herein may be utilized in other industrial applications, such as evaluating the particle size distribution for industries and technological processes in which particle size affects the physical properties of the material, for example, industries such as food processing, pharmaceuticals, cosmetics, mining, and other industries.

In some embodiments, the industrial sample may be a powdered sample or may be a sample where particles are suspended in liquid. The sample holder of the device may be configured to hold a powdered sample or a sample where particles are suspended in liquid. In some embodiments, the machine learning model may be configured to identify and classify the sizes of various particles throughout the sample. In doing so, the machine learning model may be further configured to perform a statistical analysis of the distribution of particle sizes within the sample.

### Other Use Cases

It can be appreciated that the above described use cases for the systems and methods described herein may not be exhaustive, and the systems and methods may be used and adapted as described to perform any suitable types of analyses. For example, other analyses may include: evaluation of microorganism behavior (e.g., morphology and motion) in a sample, changes in cell morphology, drug response monitoring, meningitis diagnosis, multiple sclerosis evaluation and diagnosis, prenatal biopsies such as amniocentesis and evaluation of embryo cells, or any other suitable analysis.

U.S. Provisional Patent Application. Nos. 63/622,048 and 63/715,302, to which this application claims priority, include additional details and embodiments for systems and methods for performing automated sample analysis. The provisional applications form an integral part of the instant application and may be used alone or in combination with any other embodiment described herein.

### Conclusion

Having thus described several aspects of at least one embodiment of the technology described herein, it is to be appreciated that various alterations, modifications, and improvements will readily occur to those skilled in the art. Such alterations, modifications, and improvements are intended to be part of this disclosure and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software, or a combination thereof. When implemented in software, the software code can be executed on any suitable processor or collection of processors, whether provided in a single computer or distributed among multiple computers. Such processors may be implemented as integrated circuits, with one or more processors in an integrated circuit module, including commercially available integrated circuit modules known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. A computer-readable storage medium includes any computer memory configured to store software, for example, the memory of any computing device such as a smart phone, a laptop, a desktop, a rack-mounted computer, or a server (e.g., a server storing software distributed by downloading over a network, such as an app store)). As used herein, the term “computer-readable storage medium” encompasses only a non-transitory computer-readable medium that can be considered to be a manufacture (i.e., article of manufacture) or a machine. Alternatively, or additionally, aspects of the technology described herein may be embodied as a computer readable medium other than a computer-readable storage medium, such as a propagating signal.

The terms “program” or “software” are used herein in a generic sense to refer to any type of computer code or set of processor-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor, but the processor functions may be distributed in a modular fashion among a number of different computers or processors to implement various aspects of the technology described herein.

Computer-executable instructions may be in many forms, such as program modules, executed by one or more computers or other devices. Generally, program modules include routines, programs, objects, modules, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

In some embodiments, one or more AI-based models may be used to implement one or more embodiments as described in the foregoing. Such model(s) operate generally by processing input data through one or more computational layers to produce an output. The model's architecture typically includes interconnected nodes that transform input data using learned parameters. These transformations often involve matrix multiplications, non-linear activation functions, and other mathematical operations designed to extract relevant features and patterns from the data. Generally, AI models are trained using data that is prepared and fed into the model, generating predictions and/or other outputs. Model predictions are compared to actual target values and a loss function is typically used to quantify any errors, which are back-propagated through the network while adjusting model parameters to minimize the loss. AI models can be executed on various types of processors, including CPUs and GPUs.

Various aspects of the technology described herein may be used alone, in combination, or in a variety of arrangements not specifically described in the embodiments described in the foregoing and is therefore not limited in its application to the details and arrangement of modules set forth in the foregoing description or illustrated in the drawings. For example, aspects described in one embodiment may be combined in any manner with aspects described in other embodiments.

Also, the technology described herein may be embodied as a method, of which examples are provided herein. The acts performed as part of any of the methods may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately” and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. An imaging system for automated sample analysis, the system comprising:
an imaging device configured to detect optical signals encoded with information associated with a sample, the imaging device comprising:
an emitter comprising a light source configured to illuminate the sample;
a sensor configured to detect the optical signals encoded with information associated with the sample; and
a sample holder disposed between the light source and the sensor, the sample holder configured to hold the sample and allow light to pass through from the light source to the sensor;
a processor operatively coupled with the sensor of imaging device and configured to receive data indicative of the detected optical signals and generate a plurality of images of the sample based at least in part on the detected optical signals; and
a machine learning model configured to receive the plurality of images of the sample as input from the processor and determine an output indicative of one or more attributes of the sample based at least in part on the received plurality of images of the sample.

2. The system of claim 1, wherein:
the machine learning model is trained at least in part using training images having a higher resolution than a resolution of the received plurality of images.

3. The system of claim 2, wherein:
the one or more attributes comprise at least one of: a presence of an inclusion in the sample, a size of an inclusion in the sample, a shape of an inclusion in the sample, a movement of an inclusion in the sample, and a number of an inclusion in the sample.

4. The system of claim 3, wherein:
the machine learning model is an adversarial neural network comprising:
a first portion, trained using the training images having the higher resolution, configured to generate data indicative of the one or more attributes of the sample based on the received plurality of images of the sample; and
a second portion configured to generate new data indicative of the one or more attributes of the sample based at least in part on the data generated by the first portion; and
the output is determined based at least on the data generated by the first portion and the new data generated by the second portion.

5. The system of claim 3, wherein:
the machine learning model is an adversarial neural network comprising:
a first portion, trained using the training images having the higher resolution, configured to extract image features based on the received plurality of images of the sample;
a second portion configured to generate data indicative of the one or more attributes of the sample based on the features received from the first portion; and
a third portion configured to generate new data indicative of the one or more attributes of the sample based at least in part on the data generated by the first portion; and
the output is determined based at least on the data generated by the second portion and the new data generated by the third portion.

6. The system of claim 1, wherein:
the machine learning model is a deep learning model configured to determine the one or more attributes of the sample.

7. The system of claim 1, wherein:
the machine learning model is a first machine learning model configured to determine at least a first attribute of the one or more attributes of the sample;
the system further comprises a second machine learning model configured to determine at least a second attribute of the one or more attributes of the sample; and
the output includes a first determination from the first machine learning model and a second determination from the second machine learning model.

8. The system of claim 1, wherein:
at least two of the imaging device, processor, and machine learning model are integrated on the same device.

9. The system of claim 1, wherein:
the light source is a first light source;
the emitter comprises a plurality of light sources arranged in an array; and
the emitter is configured to vary at least one illumination condition of the plurality of light sources.

10. The system of claim 9, wherein:
the optical signals detected by the imaging device are a first set of optical signals associated with a first illumination condition of the plurality of light sources; and
the imaging device is further configured to detect a second set of optical signals associated with a second illumination condition of the plurality of light sources.

11. The system of claim 10, wherein:
generating the plurality of images comprises generating a plurality of high-resolution images based at least in part on the first and second sets of optical signals.

12. The system of claim 1, wherein:
each of at least a subset of the plurality of images is associated with a respective depth of the sperm sample.

13. The system of claim 1, wherein the emitter is configured to operate in a pulse mode and operation of the emitter is configured to be synchronized with operation of the sensor.

14. The system of claim 13, wherein the operation of the emitter is synchronized with the operation of the sensor by receiving, with the emitter, one or more signals from the sensor.

15. A method for automated sample analysis, the method comprising:
detecting, with an imaging device, optical signals encoded with information associated with a sample by:
illuminating the sample using an emitter of the imaging device, the emitter disposed on a first side of the sample; and
detecting the optical signals with a sensor of the imaging device, the sensor disposed on a second side of the sample opposite the first side;
generating a plurality of images of the sample based at least in part on the optical signals; and
determining, using a machine learning model, an output indicative of one or more attributes of the sample based at least in part on the generated plurality of images of the sample.

16. The method of claim 15, wherein generating the plurality of images of the sample comprises:
determining first amplitude values and first phase values for the detected optical signals at a first plane of the sensor;
determining second amplitude values and second phase values for the detected optical signals at a second plane of the sample based on the first amplitude values and first phase values; and
updating the first amplitude values and first phase values at the first plane based on the second amplitude values and second phase values.

17. The method of claim 16, wherein generating the plurality of images further comprises:
identifying, using a second machine learning model, one or more artifacts present in the plurality of images; and
removing, using the second machine learning model, the identified one or more artifacts from the plurality of images.

18. The method of claim 15, wherein determining the output indicative of one or more attributes of the sample using the machine learning model comprises:
generating, using a first portion of the machine learning model, data indicative of the one or more attributes of the sample based on the plurality of images of the sample; and
generating, using a second portion of the machine learning model, new data indicative of the one or more attributes of the sample based at least in part on the data generated by the first portion.

19. The method of claim 15, wherein detecting the optical signals comprises:
illuminating the sample with two or more illumination conditions of the imaging device; and
detecting optical signals associated with each illumination condition of the two or more illumination conditions.

20. The method of claim 19, wherein generating the plurality of images of the sample comprises:
matching first features of the detected optical signals associated with a first illumination condition with second features of the detected optical signals associated with a second illumination condition; and
generating the plurality of images of the sample based on the matched first features and second features.
